(19) United States
(12) Patent Application Publication
(10) Pub. No.: US 2008/0312423 A1
(43) Pub. Date: Dec. 18, 2008

(54) Preparation of Metal Ion-Lactoferrin

(75) Inventors: Kay Patricia Palmano, Palmerston North (NZ); Osama Mohammad Abusidou, Palmerston North (NZ); David Francis Elgar, Palmerston North (NZ)

Correspondence Address: KNOBBE MARTENS OLSON & BEAR LLP, 2040 MAIN STREET, FOURTEENTH FLOOR, IRVINE, CA, 92614, US

(73) Assignee: FONTERRA CO-OPERATIVE GROUP LIMITED, AUCKLAND (NZ)

(21) Appl. No.: 11/916,968

(22) PCT Filed: Jun. 9, 2006
(86) PCT No.: PCT/NZ2006/000146

§ 371 Date: May 23, 2008

(30) Foreign Application Priority Data
Jun. 9, 2005 (NZ) 540633

(51) Int. Cl. C07K 14/79

(52) U.S. Cl. 530/395

(57) ABSTRACT

The present invention provides processes for preparing metal ion-lactoferrin by first immobilising lactoferrin in a lactoferrin-binding matrix and then contacting the immobilised lactoferrin with a source of metal ions, preferably a milk composition containing metal ions. A preferred product of the process is iron-lactoferrin.

### FIELD OF THE INVENTION

The present invention relates to a process for preparing metal ion-lactoferrin. More particularly the present invention relates to a process for preparing metal ion-lactoferrin by first immobilising lactoferrin in a lactoferrin-binding matrix. A preferred product of the process is iron-lactoferrin.

### BACKGROUND TO THE INVENTION

Lactoferrin is an 80 kD iron-binding glycoprotein present in most exocrine fluids, including tears, bile, bronchial mucus, gastrointestinal fluids, cervico-vaginal mucus, seminal fluid and milk. The richest source of lactoferrin is mammalian milk and colostrum. Bovine milk contains approximately 0.2 mg/ml of lactoferrin. Lactoferrin in bovine milk is approximately 12 to 18% iron saturated in its native state.

Lactoferrin has multiple postulated biological roles, including regulation of iron metabolism, immune function and embryonic development. Lactoferrin has anti-microbial activity against a range of pathogens including Gram positive and Gram negative bacteria, yeasts and fungi. The anti-microbial effect of lactoferrin is based on its capability of binding iron which is essential for the growth of the pathogens. Lactoferrin also inhibits the replication of several viruses and increases the susceptibility of some bacteria to antibiotics and lysozyme.

Bovine lactoferrin is composed of a single polypeptide chain with 17 disulfide bridges. The three-dimensional structure of bovine lactoferrin comprises two lobes (the N-lobe and C-lobe) of equal size. Each lobe comprises a metal ion-binding pocket; each pocket has the capacity to bind reversibly one Fe3+ ion with high affinity in cooperation with a CO3− ion (Moore et al, 1997). Lactoferrin in bovine milk is naturally about 12 to 18 percent iron saturated. Lactoferrin that is iron saturated to a greater extent has been reported to be useful as an iron supplement or as part of a cancer therapy regime (International Application WO 2006/054908).

Many different techniques have been reported for isolating lactoferrin from mammalian milk, many of which involve cation exchange of skim milk. See for example European patent application EP 1 466 923.

Equally, several techniques have been reported for preparing iron-saturated lactoferrin (iron-lactoferrin). These generally involved contacting substantially pure lactoferrin with an iron donor (such as an iron salt, typically a ferric salt) and a source of carbonate anions in an aqueous solution. See for example U.S. Pat. No. 5,606,086. Either dialysis or ultrafiltration (UF) and diafiltration (DF) steps are usually required to remove any excesses of iron and reagent from the iron-lactoferrin solution.

Disadvantages with reported techniques of preparing iron-lactoferrin include that ferric salts may be toxic, an additional step to remove excess iron is often required, the iron-rich permeate from the UF/DF step is a waste stream requiring disposal, the presence of iron and its chelators in waste streams or resulting products present biological and environmental problems, and reported processes may be difficult to scale-up.

Accordingly, it is an objection of the present invention to provide an improved or alternative process for preparing metal ion-lactoferrin that at least provides the public with a useful choice.

### SUMMARY OF THE INVENTION

Accordingly, one aspect of the present invention provides a process for preparing metal ion-lactoferrin comprising contacting a lactoferrin source with a lactoferrin-binding matrix to immobilise lactoferrin in the matrix, contacting the immobilised lactoferrin with a source of metal ions to produce metal ion-lactoferrin and recovering the metal ion-lactoferrin from the matrix.

Another aspect of the present invention provides a process for preparing metal ion-lactoferrin comprising

(a) contacting a column having a column volume and comprising a lactoferrin-binding matrix with a lactoferrin source to immobilise the lactoferrin in the matrix,
(b) contacting the immobilised lactoferrin with a source of metal ions to produce metal ion-lactoferrin, and
(c) recovering the metal ion-lactoferrin from the matrix.

Another aspect of the present invention provides a process for preparing metal ion-lactoferrin comprising contacting a lactoferrin source with a lactoferrin-binding matrix to immobilise lactoferrin in the matrix, contacting the immobilised lactoferrin with a source of metal ions and a milk composition in any order to produce metal ion-lactoferrin and recovering the metal ion-lactoferrin from the matrix.

Another aspect of the present invention provides a process for preparing metal ion-lactoferrin comprising

(a) contacting a column having a column volume and comprising a lactoferrin-binding matrix with a lactoferrin source to immobilise the lactoferrin in the matrix,
(b) contacting the immobilised lactoferrin with a source of metal ions and contacting the immobilised lactoferrin with a milk composition in any order to produce metal ion-lactoferrin, and
(c) recovering the metal ion-lactoferrin from the matrix.

Another aspect of the present invention provides a process for preparing metal ion-lactoferrin comprising contacting a lactoferrin source with a lactoferrin-binding matrix to immobilise lactoferrin in the matrix, contacting the immobilised lactoferrin with a milk composition comprising a source of metal ions to produce metal ion-lactoferrin and recovering the metal ion-lactoferrin from the matrix.

Another aspect of the present invention provides a process for preparing metal ion-lactoferrin comprising

(a) contacting a column having a column volume and comprising a lactoferrin-binding matrix with a lactoferrin source to immobilise the lactoferrin in the matrix,
(b) contacting the immobilised lactoferrin with at least one column volume or part thereof of a milk composition comprising a source of metal ions to produce metal ion-lactoferrin, and
(c) recovering the metal ion-lactoferrin from the matrix.

Another aspect of the present invention provides a process for preparing metal ion-lactoferrin comprising contacting a lactoferrin-binding matrix with milk or a derivative thereof containing lactoferrin to immobilise the lactoferrin in the matrix, contacting the immobilised lactoferrin with a milk composition comprising a source of metal ions to produce metal ion-lactoferrin and recovering the metal ion-lactoferrin from the matrix.

Another aspect of the present invention provides a process for preparing metal ion-lactoferrin comprising

(a) contacting a column having a column volume and comprising a lactoferrin-binding matrix with milk or a derivative thereof containing lactoferrin to immobilise the lactoferrin in the matrix,
(b) contacting the immobilised lactoferrin with at least one column volume or part thereof of the milk or derivative thereof that further comprises a source of metal ions to produce metal ion-lactoferrin, and
(c) recovering the metal ion-lactoferrin from the matrix.

Another aspect of the present invention provides a process for preparing metal ion-lactoferrin comprising

(a) contacting a membrane, optionally comprising a lactoferrin-binding matrix with a lactoferrin source to immobilise the lactoferrin on the membrane or in the matrix,
(b) contacting the immobilised lactoferrin with a source of metal ions to produce metal ion-lactoferrin, and
(c) recovering the metal ion-lactoferrin from the membrane or matrix.

Another aspect of the present invention provides a process for preparing metal ion-lactoferrin comprising

(a) contacting a membrane, optionally comprising a lactoferrin-binding matrix with milk or a derivative thereof containing lactoferrin to immobilise the lactoferrin on the membrane or in the matrix,
(b) contacting the immobilised lactoferrin with an amount of the milk or derivative thereof that further comprises a source of metal ions to produce metal ion-lactoferrin, and
(c) recovering the metal ion-lactoferrin from the membrane or matrix.

Another aspect of the present invention provides a process for preparing metal ion-lactoferrin comprising contacting a lactoferrin source with a cation exchange or affinity matrix to immobilise lactoferrin in the matrix, contacting the immobilised lactoferrin with a source of metal ions to produce metal ion-lactoferrin and recovering the metal ion-lactoferrin from the matrix.

Another aspect of the present invention provides a process for preparing metal ion-lactoferrin comprising

(a) contacting a column having a column volume and comprising a cation exchange or affinity matrix with milk or a derivative thereof containing lactoferrin to immobilise the lactoferrin in the matrix,
(b) contacting the immobilised lactoferrin with at least one column volume or part thereof of the milk or derivative thereof that further comprises a source of metal ions to produce metal ion-lactoferrin, and
(c) eluting the metal ion-lactoferrin from the matrix using a solution with a salt concentration of at least about 0.4 M, and preferably having a salt concentration of about 0.45 M, 0.5 M, 1 M, 1.5 M or 2 M.

Another aspect of the present invention provides a process for preparing metal ion-lactoferrin comprising contacting a lactoferrin-binding matrix with milk or a derivative thereof containing lactoferrin to immobilise the lactoferrin in the matrix, contacting the immobilised lactoferrin with a source of metal ions to produce metal ion-lactoferrin and recovering the metal ion-lactoferrin from the matrix.

The following embodiments may relate to any of the above aspects of the invention.

The lactoferrin source may be an aqueous composition or milk or a derivative thereof. In one embodiment the lactoferrin source is selected from the group comprising a substantially pure lactoferrin composition, a crude lactoferrin composition, mammalian milk, goat, pig, mouse, water buffalo, camel, yak, horse, donkey, llama, bovine or human milk, recombined or fresh whole milk, recombined or fresh skim milk, reconstituted whole or skim milk powder, skim milk concentrate, skim milk retentate, concentrated milk, buttermilk, ultrafiltered milk retentate, milk protein concentrate (MPC), milk protein isolate (MPI), calcium depleted milk protein concentrate (MPC), low fat milk, low fat milk protein concentrate (MPC), colostrum, a colostrum fraction, colostrum protein concentrate (CPC), colostrum whey, whey, whey protein isolate (WPI), whey protein concentrate (WPC), sweet whey, lactic acid whey, mineral acid whey, salt whey, reconstituted whey powder, any milk or colostrum processing stream comprising whey proteins, the retentate or permeate comprising whey proteins obtained by ultrafiltration or microfiltration of any milk or colostrum processing stream, or the breakthrough or adsorbed fraction comprising whey proteins obtained by chromatographic separation of any milk or colostrum processing stream.

In another embodiment the milk or derivative thereof containing lactoferrin is selected from the group comprising recombined or fresh whole milk, recombined or fresh skim milk, reconstituted whole or skim milk powder, skim milk concentrate, skim milk retentate, concentrated milk, buttermilk, ultrafiltered milk retentate, milk protein concentrate (MPC), milk protein isolate (MPI), calcium depleted milk protein concentrate (MPC), low fat milk, low fat milk protein concentrate (MPC), colostrum, a colostrum fraction, colostrum protein concentrate (CPC), colostrum whey, whey, whey protein isolate (WPI), whey protein concentrate (WPC), sweet whey, lactic acid whey, mineral acid whey, salt whey, reconstituted whey powder, any milk or colostrum processing stream comprising whey proteins, the retentate or permeate comprising whey proteins obtained by ultrafiltration or microfiltration of any milk or colostrum processing stream, or the breakthrough or adsorbed fraction comprising whey proteins obtained by chromatographic separation of any milk or colostrum processing stream.

In a further embodiment the milk composition is selected from the group comprising recombined or fresh whole milk, recombined or fresh skim milk, reconstituted whole or skim milk powder, skim milk concentrate, skim milk retentate, concentrated milk, buttermilk, ultrafiltered milk retentate, milk protein concentrate (MPC), milk protein isolate (MPI), calcium depleted milk protein concentrate (MPC), low fat milk, low fat milk protein concentrate (MPC), colostrum, a colostrum fraction, colostrum protein concentrate (CPC), colostrum whey, whey, whey protein isolate (WPI), whey protein concentrate (WPC), sweet whey, lactic acid whey, mineral acid whey, salt whey, reconstituted whey powder, derived from any milk or colostrum processing stream, derived from the retentate or permeate obtained by ultrafiltration or microfiltration of any milk or colostrum processing stream, or derived from the breakthrough or adsorbed fraction obtained by chromatographic separation of any milk or colostrum processing stream. Preferably the milk composition is skim milk, more preferably bovine skim milk.

In one embodiment the source of metal ions is a metal ion salt. Preferred salts include but are not limited to ammonium citrate, ammonium sulphate, citrate, chloride, lactate, nitrate and sulphate salts. In a preferred embodiment the source of metal ions is a ferrous salt, preferably ferrous sulphate or ammonium ferrous sulphate.

In another embodiment the metal ions include but are not limited to bismuth ions, chromium ions, cobalt ions, copper (cuprous or cupric) ions, iron (ferric or ferrous) ions, manganese ions and zinc ions, or mixtures thereof.

In one embodiment the metal ion concentration of the source of metal ions or of the milk composition is sufficient to allow stoichiometric binding of the metal ion by the immobilised lactoferrin.

In another embodiment a stoichiometric excess of metal ions is used. Preferably metal ions are used in a stoichiometric excess of at least about 2, 3, 4, 5, 6, 7, 8, 9, 10, 15, 20, 25 or 30 fold, and useful ranges may be selected between any of these preceding values (for example, from about 2 to about 10 fold, preferably 2 to about 5 fold).

In another embodiment the metal ion concentration of the source of metal ions or of the milk composition is about or at least about 0.5, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495 or 500 mg/L, and useful ranges may be selected between any of these preceding values (for example, from about 0.5 to 10, about 0.5 to 20, about 0.5 to 30, about 0.5 to 40, about 0.5 to 50, about 0.5 to 60, about 0.5 to 70, about 0.5 to 80, about 0.5 to 90, about 0.5 to 100, about 0.5 to 110, about 0.5 to 120, about 0.5 to 130, about 0.5 to 140, about 0.5 to 150, about 0.5 to 160, about 0.5 to 170, about 0.5 to 180, about 0.5 to 190, about 0.5 to 200, about 0.5 to 210, about 0.5 to 220, about 0.5 to 230, about 0.5 to 240, about 0.5 to 250, about 0.5 to 260, about 0.5 to 270, about 0.5 to 280, about 0.5 to 290, about 0.5 to 300, about 0.5 to 310, about 0.5 to 320, about 0.5 to 330, about 0.5 to 340, about 0.5 to 350, about 0.5 to 360, about 0.5 to 370, about 0.5 to 380, about 0.5 to 390 or about 0.5 to 400 mg/L).

In yet another embodiment the metal ion concentration of the milk or derivative thereof that further comprises a source of metal ions is as defined immediately above for the source of metal ions.

In another embodiment the concentration of metal ion salt in the source of metal ions or in the milk composition is about or at least about 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950 or 3000 mg/L, and useful ranges may be selected between any of these preceding values (for example, from about 100 to 200, 100 to 300, 100 to 400, 100 to 500, 100 to 600, 100 to 700, 100 to 800, 100 to 900, 100 to 1000, 100 to 1100, 100 to 1200, 100 to 1300, 100 to 1400, 100 to 1500, 100 to 1600, 100 to 1700, 100 to 1800, 100 to 1900, 100 to 2000, 100 to 2100, 100 to 2200, 100 to 2300, 100 to 2400, 100 to 2500, 100 to 2600, 100 to 2700, 100 to 2800, 100 to 2900 or 100 to 3000 mg/L).

In yet another embodiment the metal ion salt concentration of the milk or derivative thereof that further comprises a source of metal ions is as defined immediately above for the source of metal ions.

In a further embodiment the molar ratio of immobilised lactoferrin to metal ions is about or at least about 1:0.5, 1:1, 1:1.5, 1:2, 1:2.5, 1:3, 1:3.5, 1:4, 1:4.5, 1:5, 1:5.5, 1:6, 1:7, 1:8, 1:9, 1:10, 1:11, 1:12, 1:13, 1:14, 1:15, 1:6, 1:17, 1:18, 1:19, 1:20, 1:21 or 1:22 or more, and useful ranges may be selected between any of these preceding values (for example, from about 1:1 to 1:2, about 1:1 to 1:3, about 1:1 to 1:4, about 1:1 to 1:5, about 1:1 to 1:6, about 1:2 to 1:6, about 1:1 to 1:7, about 1:1 to 1:8, about 1:1 to 1:9, about 1:1 to 1:10, about 1:1 to 1:11, about 1:1 to 1:12, about 1:1 to 1:13, about 1:1 to 1:14, about 1:1 to 1:15, about 1:1 to 1:16, about 1:1 to 1:17, about 1:1 to 1:18, about 1:1 to 1:19, about 1:1 to 1:20, about 1:1 to 1:21, or about 1:1 to 1:22).

In one embodiment the at least one column volume or part thereof comprises about 0.05, 0.1, 0.25, 0.5, 0.75, 1, 2, 3, 4, 5, 6, 7, 8, 9 or 10 column volumes, and useful ranges may be selected between any of these preceding values (for example, from about 0.5 to 1, about 0.5 to 2, about 0.5 to 3, about 0.5 to 4, about 0.5 to 5, about 0.5 to 6, about 0.5 to 7, about 0.5 to 8, about 0.5 to 9 or about 0.5 to 10 column volumes).

In one embodiment the lactoferrin-binding matrix is selected from the group comprising but not limited to an ion exchange matrix, an anion exchange matrix, a weak anion exchange matrix, a cation exchange matrix, a weak cation exchange matrix, a strong cation exchange matrix, an affinity matrix, a heparin affinity matrix, a beta-lactoglobulin affinity matrix, an antibody affinity matrix, a metal ion-affinity matrix, a lactoferrin-ligand affinity matrix, an exclusion matrix (including but not limited to a gel permeation matrix and zeolite), a mixed-mode matrix (including but not limited to combinations of affinity, ion exchange, gel permeation and hydrophobic interaction matrices), a microfiltration membrane and an ultrafiltration membrane.

In one embodiment the cation exchange matrix is a strong cation exchange matrix, a weak cation exchange, a sulphonated polysaccharide matrix or a sulphonated agarose matrix.

In one embodiment the lactoferrin source or the milk or milk derivative containing lactoferrin is contacted with matrix at a flow rate of about or at least about 0.1 column volumes per hour (cv/hr). Preferably the flow rate is about 0.5 to 20 cv/hr. In another embodiment the source of metal ions or the milk composition comprising a source of metal ions or the milk or derivative thereof comprising a source of metal ions is contacted with the immobilised lactoferrin at a flow rate of about or at least about 0.1 cv/hr. Preferably the flow rate is about 0.25 to 10 cv/hr.

Other useful flow rates for all embodiments include 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25, 15.5, 15.75, 16, 16.25, 16.5, 16.75, 17, 17.25, 17.5, 17.75, 18, 18.25, 18.5, 18.75, 19, 19.25, 19.5, 19.75 or 20 column volumes per hour (cv/hr), and useful ranges may be selected between any of these preceding values (for example, from about 0.1 to 0.25, about 0.1 to 0.5, about 0.1 to 0.75, about 0.1 to 1, about 0.1 to 1.25, about 0.1 to 1.5, about 0.1 to 1.75, about 0.1 to 2, about 0.1 to 2.25, about 0.1 to 2.5, about 0.1 to 2.75, about 0.1 to 3, about 0.1 to 3.25, about 0.1 to 3.5, about 0.1 to 3.75, about 0.1 to 4, about 0.1 to 4.25, about 0.1 to 4.5, about 0.1 to 4.75, about 0.1 to 5, about 0.1 to 5.25, about 0.1 to 5.5, about 0.1 to 5.75, about 0.1 to 6, about 0.1 to 6.25, about 0.1 to 6.5, about 0.1 to 6.75, about 0.1 to 7, about 0.1 to 7.25, about 0.1 to 7.5, about 0.1 to 7.75, about 0.1 to 8, about 0.1 to 8.25, about 0.1 to 8.5, about 0.1 to 8.75, about 0.1 to 9, about 0.1 to 9.25, about 0.1 to 9.5, about 0.1 to 9.75 or about 0.1 to 10 cv/hr).

In another embodiment the volume of the source of metal ions or milk composition comprising a source of metal ions is about or at least about 0.1, 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75 or 10 column volumes, and useful ranges may be selected between any of these preceding values (for example, from about 0.1 to 0.25, about 0.1 to 0.5, about 0.1 to 0.75, about 0.1 to 1, about 0.1 to 1.25, about 0.1 to 1.5, about 0.1 to 1.75, about 0.1 to 2, about 0.1 to 2.25, about 0.1 to 2.5, about 0.1 to 2.75, about 0.1 to 3, about 0.1 to 3.25, about 0.1 to 3.5, about 0.1 to 3.75, about 0.1 to 4, about 0.1 to 4.25, about 0.1 to 4.5, about 0.1 to 4.75, about 0.1 to 5, about 0.1 to 5.25, about 0.1 to 5.5, about 0.1 to 5.75, about 0.1 to 6, about 0.1 to 6.25, about 0.1 to 6.5, about 0.1 to 6.75, about 0.1 to 7, about 0.1 to 7.25, about 0.1 to 7.5, about 0.1 to 7.75, about 0.1 to 8, about 0.1 to 8.25, about 0.1 to 8.5, about 0.1 to 8.75, about 0.1 to 9, about 0.1 to 9.25, about 0.1 to 9.5, about 0.1 to 9.75 or about 0.1 to 10 column volumes).

In one embodiment the source of metal ions (including the milk composition comprising a source of metal ions or the milk derivative comprising a course of metal ions) is recycled and contacted with the immobilised lactoferrin at least another 1, 2, 3, 4, 5, 6, 7, 8, 9 or 10 times, and useful ranges may be selected between any of these preceding values (for example, 1 to 2, 1 to 3, 1 to 4, 1 to 5, 1 to 6, 1 to 7, 1 to 8, 1 to 9 or 1 to 10 times). Accordingly, in preferred embodiments the method further comprises recycling the source of metal ions at least once at the same flow rate or at a slower flow rate. Preferably the source of metal ions is recycled 1, 2, 3, 4, 5, 6, 7, 8, 9 or 10 times at a flow rate selected from those listed above. Each subsequent pass may be at a different flow rate, preferably a slower flow rate, than the pass before.

In one embodiment the membrane is a microfiltration membrane or an ultrafiltration membrane.

In one embodiment the matrix is in solution. In another embodiment the matrix is supported in a column. In another embodiment the matrix is in the form of a membrane or is supported in a membrane.

In one embodiment the metal ion-lactoferrin is iron-lactoferrin. In another embodiment the metal ion-lactoferrin is copper-lactoferrin.

In one embodiment the metal ion-lactoferrin is recovered by contacting the lactoferrin-binding matrix with a solution having a salt concentration of at least about 0.4 M, preferably a 0.45, 0.5, 1, 1.5 or 2 M salt solution. Preferred salt solutions include potassium chloride, calcium chloride and sodium chloride salt solutions, most preferably a NaCl solution. In another embodiment the method further comprises adjusting the pH of the salt solution to about pH 7.0, 7.5, 8.0, 8.5, 9.0, 9.5, 10 or 10.5. Preferably the pH is adjusted to about pH 9 to 10.

In another embodiment the method further comprises adjusting the pH of the metal ion-lactoferrin recovered from the matrix to about pH 6.0, 6.5, 7.0, 7.5, 8.0, 8.5, 9.0, 9.5, 10 or 10.5. Preferably the pH of the recovered metal ion-lactoferrin is adjusted to about pH 6.5 to 8. Preferably the pH is adjusted by adjusting the pH of the salt solution used to elute the lactoferrin.

In one embodiment the method further comprises spray drying or freeze drying the metal ion-lactoferrin recovered from the matrix.

In one embodiment the step of contacting a column having a column volume and comprising a cation exchange matrix with milk or a derivative thereof containing lactoferrin to immobilise the lactoferrin in the matrix further comprises producing and optionally collecting a first column breakthrough composition.

In one embodiment the step of contacting the immobilised lactoferrin with at least one column volume or part thereof of the milk or derivative thereof that further comprises a source of metal ions to produce metal ion-lactoferrin further comprises producing and optionally collecting a second column breakthrough composition.

In one embodiment the first column breakthrough composition comprises the milk or derivative thereof from which lactoferrin has been removed. In one embodiment the first column breakthrough composition is substantially free of added metal ions. In another embodiment the first and second column breakthrough compositions are combined.

Another aspect of the present invention provides a metal ion-lactoferrin and a composition comprising metal ion-lactoferrin produced according to a process as defined above.

Another aspect of the present invention provides a food, drink, food additive, drink additive, dietary supplement, nutritional product, medicament, pharmaceutical or neutraceutical comprising a metal ion-lactoferrin produced according to a process as defined above.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

### DETAILED DESCRIPTION

The present invention provides in one embodiment a method for metal ion-loading of immobilised lactoferrin. In another embodiment, the present invention provides a method for metal ion-loading of immobilised lactoferrin where milk acts as the donor medium enabling exchange of metal ions into lactoferrin. In further embodiments, the present invention provides methods that allow controlled in-line metal ion-loading of lactoferrin, are scaleable, and that result in a high degree of ion loading. The principles of the invention could be applied to any metal ion-binding protein, particularly proteins in the transferrin super-family, of which lactoferrin is a member. The present invention allows production of metal ion-lactoferrin where the metal ions are bound in the metal ion-binding pockets of the lactoferrin molecule, rather than aggregates where metal ions are associated with other parts of the lactoferrin molecule.

### 1. DEFINITIONS

The term “column volume” as used herein is intended to refer to a volume equivalent to the volume of the lactoferrin-binding matrix present in a chromatographic column. This term may be used interchangeably with the term “bed volume”.

The term “comprising” as used in this specification and claims means “consisting at least in part of”. When interpreting statements in this specification and claims which include that term, the features, prefaced by that term in each statement or claim, all need to be present but other features can also be present.

The term “lactoferrin” as used herein is intended to mean any native, synthetic or recombinant lactoferrin molecule or fragment thereof that includes at least one metal ion binding pocket including but not limited to sheep, goat, pig, mouse, water buffalo, camel, yak, horse, donkey, llama, bovine or human lactoferrin and any metal ion binding fragment thereof including but not limited to N-lobe and C-lobe fragments. Full length native lactoferrin has two metal ion-binding pockets and the N-lobe and C-lobe lactoferrin fragments each have one metal ion-binding pocket. (Moore et al, 1997)

In one embodiment the lactoferrin is recombinant lactoferrin or a recombinant lactoferrin fragment. Recombinant lactoferrin and lactoferrin fragments can be produced in cells including bacterial, yeast, animal and plant cells. Production of lactoferrin in bacterial, yeast and animal cells is reported in U.S. Pat. No. 5,571,691 and U.S. Pat. No. 6,228,614. Production of recombinant fragments is reported by Tanaka et al (2003). A preferred recombinant lactoferrin is recombinant human or bovine lactoferrin.

In one embodiment the lactoferrin is apo-lactoferrin. In another embodiment the lactoferrin is iron saturated at native levels of approximately 12 to 18%. In another embodiment the lactoferrin is partially metal-ion saturated. For example, recombinant lactoferrin may have a higher degree of metal ion-saturation, usually iron ion-saturation and the method of the present invention may be used to increase the degree of metal ion-saturation.

The term “lactoferrin-binding matrix” as used herein is intended to mean a matrix that is preferably able to bind lactoferrin but includes a matrix that is at least able to retard the progress of lactoferrin when lactoferrin comes into contact with it. In one embodiment the lactoferrin-binding matrix is selected from the group comprising but not limited to an ion exchange matrix, an anion exchange matrix, a weak anion exchange matrix, a cation exchange matrix, a weak cation exchange matrix, a strong cation exchange matrix, an affinity matrix, a heparin affinity matrix (a matrix where heparin is immobilised and available to bind lactoferrin), a beta-lactoglobulin affinity matrix, an antibody affinity matrix, a metal-ion affinity matrix, a lactoferrin-ligand affinity matrix, an exclusion matrix (including but not limited to a gel permeation matrix and a zeolite), a mixed-mode matrix (including but not limited to combinations of affinity, ion exchange, gel permeation and hydrophobic interaction matrices), a microfiltration membrane and an ultrafiltration membrane.

The terms “metal ion-lactoferrin” and “metal ion-saturated lactoferrin” as used herein are intended to refer to a population of lactoferrin molecules where at least about 25% of the metal ion-binding pockets present in the population have a metal ion bound. Lactoferrin has two metal-ion binding pockets and so can bind metal ions in a stoichiometric ratio of 2 metal ions per lactoferrin molecule. Preferably at least about 25, 30, 35, 40, 45, 50, 55, 60, 65, 70, 75, 80, 85, 90, 95 or 99% of the metal ion-binding pockets present in the population have a metal ion bound, as determined by spectrophotometric analysis (Brock & Azabe, 1976; Bates et al, 1967; Bates et al, 1973). Preferably at least about 40% of the metal ion-binding pockets present in the population have a metal ion bound. It should be understood that there may be metal ion-exchange between lactoferrin molecules. Where iron is correctly bound into the iron-binding pockets, with no excess of iron, the UV-Visible spectra show a well defined absorption peak with a maximum at or very close to 465 nm. Where there is excess iron, either complexed non-specifically to lactoferrin or in solution, the absorption peak becomes distorted and there is no clear maximum at 465 nm.

In one embodiment the source of metal ions is a metal ion salt. Preferred salts include but are not limited to ammonium citrate, ammonium sulphate, citrate, chloride, lactate, nitrate and sulphate salts. In a preferred embodiment the source of metal ions is a ferrous salt, preferably ferrous sulphate.

In one embodiment the metal ions include but are not limited to bismuth ions, chromium ions, cobalt ions, copper (cuprous or cupric) ions, iron (ferric or ferrous) ions, manganese ions and zinc ions.

In one embodiment the metal ion-lactoferrin is iron-lactoferrin.

The terms “iron-lactoferrin” and “iron-saturated lactoferrin” as used herein are intended to mean that at least about 25, 30, 35, 40, 45, 50, 55, 60, 65, 70, 75, 80, 85, 90, 95 or 99% of the metal ion-binding pockets have an iron ion bound.

The term “milk derivative” as used herein, for example in the phrase “milk or derivative thereof”, is intended to refer to milk that has been processed or fractionated in some way by known techniques. Such known techniques are described in the Dairy Processing Handbook (Tetra Pak Processing Systems, Lund, Sweden, 1995); the preparation of skim milk for example. Preferred milk derivatives may be selected from the group comprising but not limited to recombined whole milk, recombined or fresh skim milk, reconstituted whole or skim milk powder, skim milk concentrate, skim milk retentate, concentrated milk, buttermilk, ultrafiltered milk retentate, milk protein concentrate (MPC), milk protein isolate (MPI), calcium depleted milk protein concentrate (MPC), low fat milk, low fat milk protein concentrate (MPC), colostrum, a colostrum fraction, colostrum protein concentrate (CPC), colostrum whey, whey, whey protein isolate (WPI), whey protein concentrate (WPC), sweet whey, lactic acid whey, mineral acid whey, salt whey, reconstituted whey powder, derived from any milk or colostrum processing stream, derived from the retentate or permeate obtained by ultrafiltration or microfiltration of any milk or colostrum processing stream, or derived from the breakthrough or adsorbed fraction obtained by chromatographic separation of any milk or colostrum processing stream. If lactoferrin is not present in the milk derivative, it may be added by incorporating a lactoferrin source including pure lactoferrin or milk or a milk derivative containing lactoferrin.

The term “strong cation exchange” as used herein is intended to refer to a strongly acidic cation exchange matrix including but not limited to sulphonated, sulphomethyl, sulphoethyl and sulphopropyl matrices (SP Sepharose Big Beads™ and SP Sephadex™ cation exchange resins for example).

The term “weak cation exchange” as used herein is intended to refer to a weakly acidic cation exchange matrix including but not limited to carboxy or carboxy-methyl substituted matrix (CM Sephadex™ and CM Sepharose™ cation exchange resins for example).

### 2. PRODUCTION OF METAL ION-LACTOFERRIN

In a broad general aspect the present invention provides a process for preparing metal ion-lactoferrin comprising contacting a lactoferrin source with a lactoferrin-binding matrix to immobilise lactoferrin in the matrix, contacting the immobilised lactoferrin with a source of metal ions to produce metal ion-lactoferrin and recovering the metal ion-lactoferrin from the matrix.

When a column-based system is used, the process of this embodiment may comprise contacting a column having a column volume and comprising a lactoferrin-binding matrix with a lactoferrin source to immobilise the lactoferrin in the matrix.

Within the scope of this broad general aspect, the lactoferrin source and the source of metal ions may be independently selected from an aqueous source and a milk source. If aqueous sources are used, in one embodiment a washing step may be included. That is, before or after the lactoferrin immobilisation step or before or after the metal ion loading step, or any combination thereof, the column may be contacted with a wash solution. In some embodiments the wash solution is an aqueous solution such as a buffer solution. Preferred buffer solutions may contain sources of citrate or carbonate ions or mixtures thereof. In other embodiments the wash solution is a milk composition. In a preferred embodiment the wash solution comprises at least a partial column volume of milk or a derivative thereof. Preferably at least one column volume of milk is used for the wash step.

Therefore, in one embodiment a process for preparing metal ion-lactoferrin comprises contacting a lactoferrin source with a lactoferrin-binding matrix to immobilise lactoferrin in the matrix, contacting the immobilised lactoferrin with a source of metal ions and a milk composition in any order to produce metal ion-lactoferrin and recovering the metal ion-lactoferrin from the matrix. The lactoferrin source and the source of metal ions may be independently selected from an aqueous source and a milk source.

The source of metal ions and milk composition may be contacted with the immobilised lactoferrin in any order. Preferably the source of metal ions is contacted with the immobilised lactoferrin first, followed by the milk composition.

In another embodiment a process for preparing metal ion-lactoferrin comprises contacting a lactoferrin source with a lactoferrin-binding matrix to immobilise lactoferrin in the matrix, contacting the immobilised lactoferrin with a milk composition comprising a source of metal ions to produce metal ion-lactoferrin and recovering the metal ion-lactoferrin from the matrix.

When a column-based system is used, the process of this embodiment may comprise

(a) contacting a column having a column volume and comprising a lactoferrin-binding matrix with a lactoferrin source to immobilise the lactoferrin in the matrix,
(b) contacting the immobilised lactoferrin with at least one column volume or part thereof of a milk composition comprising a source of metal ions to produce metal ion-lactoferrin, and
(c) recovering the metal ion-lactoferrin from the matrix.

The lactoferrin source may be an aqueous source or a milk source.

In another embodiment a process for preparing metal ion-lactoferrin comprises contacting a lactoferrin-binding matrix with milk or a derivative thereof containing lactoferrin to immobilise the lactoferrin in the matrix, contacting the immobilised lactoferrin with a milk composition comprising a source of metal ions to produce metal ion-lactoferrin and recovering the metal ion-lactoferrin from the matrix.

When a column-based system is used, the process of this embodiment may comprise

(a) contacting a column having a column volume and comprising a lactoferrin-binding matrix with milk or a derivative thereof containing lactoferrin to immobilise the lactoferrin in the matrix,
(b) contacting the immobilised lactoferrin with at least one column volume or part thereof of the milk or derivative thereof that further comprises a source of metal ions to produce metal ion-lactoferrin, and
(c) recovering the metal ion-lactoferrin from the matrix.

The source of metal ions may be added to a milk composition prepared off-line from the main processing stream or the source of metal ions may be added in-line to the milk stream being loaded onto the column.

As an alternative to a column-based system, a membrane-based system may be used. Accordingly, in another embodiment a process for preparing metal ion-lactoferrin comprises

(a) contacting a membrane, optionally comprising a lactoferrin-binding matrix with a lactoferrin source to immobilise the lactoferrin on the membrane or in the matrix,
(b) contacting the immobilised lactoferrin with a source of metal ions to produce metal ion-lactoferrin, and
(c) recovering the metal ion-lactoferrin from the membrane or matrix.

As described above, the lactoferrin source and the source of metal ions may be independently selected from an aqueous source and a milk source.

Where the lactoferrin source is a milk source, one embodiment of the process comprises

(a) contacting a membrane, optionally comprising a lactoferrin-binding matrix with milk or a derivative thereof containing lactoferrin to immobilise the lactoferrin on the membrane or in the matrix,
(b) contacting the immobilised lactoferrin with an amount of the milk or derivative thereof that further comprises a source of metal ions to produce metal ion-lactoferrin, and
(c) recovering the metal ion-lactoferrin from the membrane or matrix.

The lactoferrin binding matrix is preferably an ion exchange or affinity matrix, as described above. In one embodiment a process for preparing metal ion-lactoferrin comprises

(a) contacting a column having a column volume and comprising a cation exchange matrix or a heparin affinity matrix with milk or a derivative thereof containing lactoferrin to immobilise the lactoferrin in the matrix,
(b) contacting the immobilised lactoferrin with at least one column volume or part thereof of the milk or derivative thereof that further comprises a source of metal ions to produce metal ion-lactoferrin, and
(c) eluting the metal ion-lactoferrin from the matrix using a solution with a salt concentration of at least about 0.4 M.

Preferred salt solutions include potassium chloride, calcium chloride and sodium chloride salt solutions. Most preferably the salt solution is a NaCl solution.

In another preferred embodiment a process for preparing metal ion-lactoferrin comprises contacting a lactoferrin-binding matrix with milk or a derivative thereof containing lactoferrin to immobilise the lactoferrin in the matrix, contacting the immobilised lactoferrin with a source of metal ions to produce metal ion-lactoferrin and recovering the metal ion-lactoferrin from the matrix.

In another preferred embodiment a process for preparing metal ion-lactoferrin comprises mixing a lactoferrin source with a milk composition comprising a source of metal ions to produce metal ion-lactoferrin, contacting the mixture with a lactoferrin-binding matrix to immobilise the metal ion-lactoferrin in the matrix and recovering the metal ion-lactoferrin from the matrix.

In an exemplary embodiment lactoferrin is immobilised in a cation exchange matrix by contacting a lactoferrin source with the cation exchange matrix. The cation exchange matrix may be loaded into a column before or after it is contacted with the lactoferrin source. The immobilised lactoferrin is then contacted with a source of metal ions, preferably in the form of a metal salt, preferably a metal sulphate. The source of metal ions is preferably added to the final load volume (or part thereof) of the lactoferrin source before it is contacted with the cation exchange matrix. In the case of a cation exchange matrix loaded into a column, the source of metal ions is preferably added to the final column volume (or part thereof) of the lactoferrin source prior to its passage through the column. The final load volume (or part thereof) may be cycled multiple times through the matrix or column.

The breakthrough lactoferrin source from this last load volume may be added back to the initial breakthrough lactoferrin source (from initial lactoferrin loading) with little effect on the iron levels of the lactoferrin source. In some embodiments where stoichiometric amounts of metal ion are used, the method results in an iron-lactoferrin that may be eluted from the matrix or column and a substantially uncontaminated column breakthrough.

Accordingly, in one embodiment the step of contacting a column having a column volume and comprising a cation exchange matrix with milk or a derivative thereof containing lactoferrin to immobilise the lactoferrin in the matrix further comprises producing and optionally collecting a first column breakthrough composition.

In another embodiment the step of contacting the immobilised lactoferrin with at least one column volume or part thereof of the milk or derivative thereof that further comprises a source of metal ions to produce metal ion-lactoferrin further comprises producing and optionally collecting a second column breakthrough composition.

In one embodiment the first column breakthrough composition comprises the milk or derivative thereof from which lactoferrin has been removed. In one embodiment the first column breakthrough composition is substantially free of added metal ions.

In another embodiment the first and second column breakthrough compositions are combined. In one embodiment the metal ion concentration of the combined column breakthrough compositions is substantially the same as the metal ion concentration of the milk or derivative thereof.

In one embodiment a highly preferred process of the invention comprises immobilisation of lactoferrin from milk, preferably bovine milk onto a column having a column volume and comprising a cation exchange matrix, addition of iron as a ferrous salt, preferably ferrous sulphate to the final column volume (or part thereof) of milk prior to its passage through the column, then passage (or multiple cycling) of this final column volume (or part thereof) through the column.

In one embodiment the at least one column volume or part thereof comprises at least about 0.05, 0.1, 0.25, 0.5, 0.75, 1, 2, 3, 4, 5, 6, 7, 8, 9 or 10 column volumes, and useful ranges may be selected between any of these preceding values (for example, from about 1 to about 4 column volumes).

As discussed above, lactoferrin has two metal-ion binding pockets and so can bind metal ions in a stoichiometric ratio of 2 metal ions per lactoferrin molecule. Thus, in one embodiment the method comprises stoichiometric loading of metal ions, preferably iron ions into lactoferrin. This results in virtually all the metal ions being exchanged into the lactoferrin. This has the added benefit of preventing contamination of the lactoferrin-binding matrix used and cross contamination of the next product passed through the matrix.

In one embodiment the metal ion concentration of the source of metal ions or of the milk composition is sufficient to allow stoichiometric binding of the metal ion by the immobilised lactoferrin.

In another embodiment a stoichiometric excess of metal ions is used. Preferably metal ions are used in a stoichiometric excess of at least about 2, 3, 4, 5, 6, 7, 8, 9, 10, 15, 20, 25 or 30 fold, and useful ranges may be selected between any of these preceding values (for example, from about 2 to about 10 fold, preferably 2 to about 5 fold). That is, at least about twice as many metal ions are provided than would be required to fully saturate every molecule of lactoferrin with 2 metal ions.

In one embodiment the metal ion or metal ion salt concentration of the source of metal ions, the milk composition or the milk derivative is as defined above.

In one embodiment cycling of the iron-laden final load volume of milk (or part thereof) through the matrix or column may be carried out to obtain very high saturation levels (greater than 85%), especially if only slight excesses of iron are used.

In alternative embodiments lactoferrin is adsorbed/immobilised from any lactoferrin source (including milk, simulated milk, milk permeate or water) onto any supporting matrix (including ion-exchange resins and affinity resins) for iron loading.

The lactoferrin source is preferably a substantially pure lactoferrin composition, a crude lactoferrin composition or mammalian milk. Preferably the milk is sheep, goat, pig, mouse, water buffalo, camel, yak, horse, donkey, llama, bovine or human milk. Preferably the milk is bovine milk.

Preferably the lactoferrin source is recombined or fresh whole milk, recombined or fresh skim milk, reconstituted whole or skim milk powder, skim milk concentrate, skim milk retentate, concentrated milk, buttermilk, ultrafiltered milk retentate, milk protein concentrate (MPC), milk protein isolate (MPI), calcium depleted milk protein concentrate (MPC), low fat milk, low fat milk protein concentrate (MPC), colostrum, a colostrum fraction, colostrum protein concentrate (CPC), colostrum whey, whey, whey protein isolate (WPI), whey protein concentrate (WPC), sweet whey, lactic acid whey, mineral acid whey, salt whey, reconstituted whey powder, derived from any milk or colostrum processing stream, derived from the retentate or permeate obtained by ultrafiltration or microfiltration of any milk or colostrum processing stream, or derived from the breakthrough or adsorbed fraction obtained by chromatographic separation of any milk or colostrum processing stream.

Where the lactoferrin source comprises fat it is preferably first subjected to a filtration step to substantially remove the fat.

Preferably the final load volume is the same as the lactoferrin source or is separately selected from recombined or fresh whole milk, recombined or fresh skim milk, reconstituted whole or skim milk powder, skim milk concentrate, skim milk retentate, concentrated milk, buttermilk, ultrafiltered milk retentate, milk protein concentrate (MPC), milk protein isolate (MPI), calcium depleted milk protein concentrate (MPC), low fat milk, low fat milk protein concentrate (MPC), colostrum, a colostrum fraction, colostrum protein concentrate (CPC), colostrum whey, whey, whey protein isolate (WPI), whey protein concentrate (WPC), sweet whey, lactic acid whey, mineral acid whey, salt whey, reconstituted whey powder, derived from any milk or colostrum processing stream, derived from the retentate or permeate obtained by ultrafiltration or microfiltration of any milk or colostrum processing stream, or derived from the breakthrough or adsorbed fraction obtained by chromatographic separation of any milk or colostrum processing stream.

In one embodiment the milk composition or the milk or derivative thereof containing lactoferrin is selected from the group comprising recombined or fresh whole milk, recombined or fresh skim milk, reconstituted whole or skim milk powder, skim milk concentrate, skim milk retentate, concentrated milk, buttermilk, ultrafiltered milk retentate, milk protein concentrate (MPC), milk protein isolate (MPI), calcium depleted milk protein concentrate (MPC), low fat milk, low fat milk protein concentrate (MPC), colostrum, a colostrum fraction, colostrum protein concentrate (CPC), colostrum whey, whey, whey protein isolate (WPI), whey protein concentrate (WPC), sweet whey, lactic acid whey, mineral acid whey, salt whey, reconstituted whey powder, derived from any milk or colostrum processing stream, derived from the retentate or permeate obtained by ultrafiltration or microfiltration of any milk or colostrum processing stream, or derived from the breakthrough or adsorbed fraction obtained by chromatographic separation of any milk or colostrum processing stream. Preferably the milk composition or the milk or derivative thereof is skim milk, more preferably bovine skim milk.

The small amount of iron-enriched milk produced as a by-product in some embodiments of the invention can be combined with the large volume of milk voided during commercial lactoferrin isolation. Where larger amounts of iron are used, the iron-enriched milk can be recycled in a method of the invention or incorporated into other dairy processing streams.

### 3. LACTOFERRIN-BINDING MATRICES

In a preferred embodiment of the invention, a lactoferrin-binding matrix comprising a cation exchange matrix is used.

In alternative embodiments the lactoferrin-binding matrix may be selected from the group comprising but not limited to an ion exchange matrix, an anion exchange matrix, a weak anion exchange matrix, a cation exchange matrix, a weak cation exchange matrix, a strong cation exchange matrix, an affinity matrix, a heparin affinity matrix, a beta-lactoglobulin affinity matrix, an antibody affinity matrix, a metal-ion affinity matrix, a lactoferrin-ligand affinity matrix, an exclusion matrix (including but not limited to a gel permeation matrix and zeolite), a mixed-mode matrix (including but not limited to combinations of affinity, ion exchange, gel permeation and hydrophobic interaction matrices), a microfiltration membrane and an ultrafiltration membrane.

In one embodiment the cation exchange matrix is a strong cation exchange matrix, a weak cation exchange, a sulphonated polysaccharide matrix or a sulphonated agarose matrix.

In one embodiment the membrane is a microfiltration membrane or an ultrafiltration membrane.

In one embodiment the matrix is in solution. In another embodiment the matrix is supported in a column. In another embodiment the matrix is in the form of a membrane or is supported in a membrane.

It should be understood that the elution conditions required for a particular lactoferrin-binding matrix may be readily determined by an ordinarily skilled worker with regard to that skill and the teaching of this specification, without undue experimentation.

In one embodiment the metal ion-lactoferrin is recovered by contacting the lactoferrin-binding matrix with a solution having a salt concentration of at least about 0.4 M, and preferably having a salt concentration of about 0.45 M, 0.5 M, 1 M, 1.5 M or 2 M. Preferred salt solutions include potassium chloride, calcium chloride and sodium chloride salt solutions. A highly preferred solution is a NaCl solution.

In one embodiment the process of the invention is a batch process. In another embodiment it is a continuous process.

Preferred lactoferrin-binding matrices known in the art are discussed below (see for example, Scopes, 1994).

### 3.1 Ion Exchange Chromatography

Ion exchange chromatography relies on the use of charge interactions. The choice of the ion-exchange matrix, whether anion or cation, or strong or weak, is influenced mainly by the isoelectric point of the component requiring purification and the effect of pH on its charge.

The isoelectric point (pI) of bovine lactoferrin ranges from 8.0 to 8.9 and of human lactoferrin from 5.8 to 10.0. The nature of the lactoferrin molecule chosen for metal ion-saturation, including its source will direct the choice of ion exchange system selected.

Elution conditions are selected so that the molecule of interest no longer associates with the ion exchange resin. This lack of association is usually due to a change in charge. A neutral molecule or one with the same charge as the ion exchange resin will not bind to the resin.

As discussed above, the required elution conditions may be readily determined by an ordinarily skilled worker with regard to that skill and the teaching of this specification, without undue experimentation. Preferred elution conditions for cation exchange chromatography are discussed above.

Ion exchange techniques generally are known in the art. See for example: Ion Exchange Chromatography & Chromatofocusing, Principles and Methods, Amersham Biosciences Limited 2004, Code 11-0004-21, Edition AA, http://www.amersham.com.

There are three common methods of ion exchange these are packed bed, stirred tank and expanded bed.

### Expanded Bed Adsorption

Expanded bed adsorption involves flow from the base of the column to expand the bed and allows free passage of particulate matter. This system has good kinetics and enables high flow rates without high pressure drops. It is also less expensive to implement than packed bed systems.

### Packed Bed Adsorption

Packed bed columns have broad application, high capacity, high throughput, but are limited by kinetics and structural properties. Use of a packed bed system may require large vessels and long transfer times and is most economic for solutions comprising less than 1% protein.

### Stirred Tank Adsorption

Stirred tank systems are also suitable for large samples with low protein concentrations in batch or continuous form. They can accept feed with more particulate matter than a packed bed.

### 3.2 Affinity Chromatography

Affinity chromatography exploits highly specific biological interactions between two molecules. Blackberg et al (1980) report use of a heparin-affinity matrix to isolate lactoferrin from human whey. Ena et al (1990) report use of a bovine beta-lactoglobulin affinity matrix to isolate lactoferrin.

### 3.3 Reverse Phase High Performance Liquid Chromatography

High performance liquid chromatography (HPLC) separation is predominantly driven by hydrophobic interactions of the solute with the packing. Elution of the absorbed peptide is typically accomplished by gradient elution using water-miscible organic solvents. Palmano et al (2002) report use of HPLC to isolate bovine lactoferrin from bovine whey.

### 3.4 Size Exclusion Chromatography (Gel Filtration or Gel Permeation)

Size exclusion chromatography is widely employed in biochemical research to purify small quantities of proteins based on their size difference. The separation depends on the ability of a molecule to penetrate porous particles in the stationary phase in a chromatography column.

### 3.5 Filtration

### Microfiltration

Microfiltration (MF) allows collection of entities visible through a microscope (cells, large virus particles, cellular debris) through use of membranes having pore sizes ranging from 0.05 to 10 μm in diameter.

### Ultrafiltration

Ultrafiltration (UF) membrane are classified by reference to the nominal molecular weight cut off (MWCO) which retains approximately 95% of material larger than the indicated size. UF membranes have MWCO's ranging from 100 to 1,000,000 Daltons. Any entities smaller than the rated MWCO of the membrane will usually pass through the membrane.

### Cross-Flow Filtration

The feedstock is run tangential to the membrane which creates a pressure differential across the membrane. As a result, some particles pass through the membrane. Other particles continue to flow across the membrane, “cleaning it”. In contrast to the dead-end filtration, a tangential flow will prevent thicker particles from building up a “filter cake”.

### Dead-End Filtration

All the fluid is applied perpendicularly to the membrane and all particles larger than the pore size of the membrane are retained at its surface. The trapped particles will form a “filter cake” at the surface of the membrane.

### Recovery of Metal-Ion Lactoferrin

Dead-end filtration of pre-isolated lactoferrin may be used to immobilise the lactoferrin on the membrane surface ready for metal ion loading. Metal ion-lactoferrin may be recovered off the membrane surface using cross-flow filtration with zero or slightly negative transmembrane pressure.

### 4. PRODUCTS

Another aspect of the present invention provides a metal ion-lactoferrin produced according to a process as defined above.

The metal ion-lactoferrin of the invention may be incorporated into a food, drink, food additive, drink additive, dietary supplement, nutritional product, medicament, pharmaceutical or neutraceutical. Such products are also provided by the present invention.

Various aspects of the invention will now be illustrated in non-limiting ways by reference to the following examples.

### EXAMPLE 1

### Batch Iron-Loading of Lactoferrin in Skim Milk

### Comparative Example

0.1 g FeSO₄.7H₂O (iron (II) sulphate heptahydrate, Sigma-Aldrich, New Zealand) was dissolved into 5 L skimmed milk (Fonterra Co-Operative Group Limited, New Zealand) to give a concentration of 20 mg/L. 20 mL SP Sepharose Big Beads™ cation exchange matrix (SPBB, Amersham Biosciences, Sweden) were added and the whole stirred for 4 hours. The SPBB were captured on a column, rinsed with water and adsorbed proteins eluted sequentially with 0.35M, 1.0 M and 2.0 M NaCl. The bulk of lactoferrin (Lf) was eluted in the 1.0 M eluate. Lf in both 1.0 M and 2.0 M eluates was found to be approximately 60-80% iron (Fe) loaded by spectrophotometric analysis (Brock & Azabe, 1976; Bates et al, 1967; Bates et al, 1973). Lf in the milk was estimated at approximately 1 g total. Stoichiometric excess of Fe:Lf was approximately 25 fold.

### EXAMPLE 2

### Batch Iron-Loading of Lactoferrin in Skim Milk

### Comparative Example

0.4 g FeSO₄.7H₂O (iron (II) sulphate heptahydrate, Sigma-Aldrich, New Zealand) was dissolved into 5 L skimmed milk (Fonterra Co-Operative Group Limited, New Zealand) to give a concentration of 80 mg/L. 20 mL SPBB (Amersham Biosciences, Sweden) were added and the whole stirred for 4 hours. The SPBB were captured on a column and adsorbed proteins were eluted sequentially with 0.35M, 1.0 M and 2.0 M NaCl. The bulk of Lf was eluted in the 1.0 M eluate. Lf in both 1.0 M and 2.0 M eluates was found to be approximately 80% Fe loaded by spectrophotometric analysis. Lf in the milk was estimated at approximately 1 g total. Stoichiometric excess of Fe:Lf was approximately 100 fold.

### EXAMPLE 3

### Approximately Stoichiometric Iron Loading of Immobilised Lactoferrin

0.8 g of lactoferrin (Fonterra Co-Operative Group Limited, New Zealand) was loaded onto 30 ml SPBB (Amersham Biosciences, Sweden) in a column. The column was washed with 8 column volumes of skim milk (Fonterra Co-Operative Group Limited, New Zealand) then 1 column volume of skim milk containing 4 mg FeSO₄.7H₂O (iron (II) sulphate heptahydrate, Sigma-Aldrich, New Zealand). This last column volume was cycled through the column five times. The column was washed with water and adsorbed protein eluted sequentially with 1 M NaCl and 2 M NaCl. Eluted Lf was found to be fully iron saturated (90-100%) by spectrophotometric analysis. The amount of Fe added as FeSO₄ was almost sufficient for stoichiometric (2 Fe: 1 Lf) binding by Lf.

### EXAMPLE 4

### Iron Loading of Immobilised Lactoferrin with 3 to 4 Fold Excess Iron

6 L of skimmed milk (Fonterra Co-Operative Group Limited, New Zealand) (Lf concentration approx. 0.2 mg/mL) was passed through 30 mL SPBB (Amersham Biosciences, Sweden) in a column. 40 mL of milk containing 16 mg FeSO₄.7H₂O (iron (11) sulphate heptahydrate, Sigma-Aldrich, New Zealand) was then passed though the column, the column was washed with water and the adsorbed protein eluted sequentially with 0.4 M NaCl, 1 M NaCl and 2 M NaCl. The majority of Lf was eluted in the 1 M NaCl fraction and was found to be fully Fe saturated by spectrophotometric titration. Based on the recovery of lactoferrin on the column, the amount of Fe added as FeSO₄ was estimated to be in approximately 3 fold excess of that required for stoichiometric Lf saturation. Of the Fe loaded, approximately 65% was recovered in the breakthrough milk after loading and approximately 0.6% in the subsequent water wash. The amount of iron recovered in the Fe-loaded milk which had passed through the column was approximately equal to the initial load minus the amount bound to Lf. The ratio of total Fe to Lf protein in both the 1 M NaCl and 2 M NaCl eluates was close to that expected for full saturation of Lf. Total iron in samples was measured by ICP-OES (inductively coupled plasma-optical emission spectroscopy) or ICP-MS (inductively coupled plasma-mass spectroscopy) (Association of Official Analytical Chemists, 16th Edition, Method 984.27). Lf was measured by reversed-phase HPLC (Elgar et al., 2000; Palmano et al, 2002).

### EXAMPLE 5

### Iron Loading of Immobilised Lactoferrin with 2.5 Fold Excess Iron

30 mL of SPBB (Amersham Biosciences, Sweden) in a column (resin height 20 cm) was loaded with 1 g of Lf (Fonterra Co-Operative Group Limited, New Zealand) dissolved in 60 mL of 0.3 M NaCl. The column was washed with 40 mL of skimmed milk (Fonterra Co-Operative Group Limited, New Zealand) then another 40 mL of skimmed milk in which 6 mg of FeSO₄.7H₂O (iron (II) sulphate heptahydrate, Sigma-Aldrich, New Zealand) had been dissolved was passed through. There was an instant colour change on the column from pale pink to dark pink. Another 6 mg of FeSO₄ in 20 mL of skimmed milk was passed through the column with no further apparent change in colour. The column was washed with water and the bound protein eluted sequentially with 0.4 M NaCl and 2 M NaCl. Lf was recovered in the 2 M NaCl eluate and was found to be >80% Fe-saturated by spectrophotometric titration. The amount of Fe added as FeSO₄ was estimated to be approximately 2.5 fold that required for stoichiometric Lf saturation.

### EXAMPLE 6

### Iron Loading of Immobilised Lactoferrin with 20 Fold Excess Iron

1 g Lf (Fonterra Co-Operative Group Limited, New Zealand) was dissolved in 60 mL of skimmed milk (Fonterra Co-Operative Group Limited, New Zealand) and passed through a 30 mL column of SPBB (Amersham Biosciences, Sweden). 60 mL water containing 95 mg of FeSO₄.7H₂O (iron (II) sulphate heptahydrate, Sigma-Aldrich, New Zealand) was immediately passed through the column. The column was washed with water and adsorbed protein eluted sequentially with 1 M NaCl and 2 M NaCl. The majority of Lf eluted in the 1 M NaCl fraction and appeared to be almost fully Fe saturated by spectrophotometric titration and visible observation of colour. The presence of excess Fe in the eluate made precise estimations of saturation difficult. The amount of Fe added as FeSO₄ was estimated to be approximately 20 fold that required for stoichiometric Lf saturation. Of the Fe loaded, approximately 4% was recovered in the wash water following Fe loading, with the majority being recovered in the 1 M NaCl eluate. The stoichiometric excess of Fe:Lf in this eluate was estimated to be approximately 14 fold.

### EXAMPLE 7

### Iron Loading of Immobilised Lactoferrin with 10 to 12 Fold Excess Iron

1 g Lf (Fonterra Co-Operative Group Limited, New Zealand) was dissolved in 80 mL skimmed milk (Fonterra Co-Operative Group Limited, New Zealand) and passed through a 30 mL column of SPBB (Amersham Biosciences, Sweden). The column was washed with water and 50 mg FeSO₄.7H₂O (iron (II) sulphate heptahydrate, Sigma-Aldrich, New Zealand) in 50 mL water cycled through the column 3 times. The column was washed with water and adsorbed protein eluted sequentially with 1 M NaCl and 2 M NaCl. The majority of the Lf was eluted in the 1 M NaCl fraction and appeared to be almost fully Fe saturated by spectrophotometric titration and visible observation of colour. The presence of excess Fe in the eluate made precise estimations of saturation difficult. The amount of Fe added as FeSO₄ was estimated to be in approximately 10-12 fold excess of that required for stoichiometric Lf saturation.

Of the Fe loaded, approximately 0.4% was recovered in the load water after 3 passages through the column, approximately 81% was recovered in the 1 M NaCl eluate and approximately 4% was recovered in the 2 M NaCl eluate. The stoichiometric excess of Fe:Lf in the 1 M NaCl eluate was estimated to be approximately 10 fold.

### EXAMPLE 8

### Batch Iron-Loading of Lactoferrin in Skim Milk

### Comparative Example

0 mg, 5 mg or 10 mg of ammonium ferrous sulphate was added to 650 mL aliquots of fresh skim milk (Fonterra Co-operative Group) and gently stirred by magnetic flea for 2 h at RT. 18 g (˜20 mL) of SPBB pre-swollen in milliQ water added with further stirring for 4 h at RT. The resin was recovered on a sintered funnel, the breakthrough milk (non-bound fraction) collected and the resin washed with milliQ water, followed by 40 mL 50 mM disodium phosphate/0.15M sodium chloride pH 7.0 (Buffer A). Lactoferrin was eluted from the resin with 1.5M sodium chloride. The lactoferrin eluates were dialysed against milliQ water overnight at 4° C. using 3,500 molecular weight cut-off tubing. The dialysed lactoferrin eluates were then loaded at 1.5 mL/min onto a 20 mL column (1.6 cm×15 cm) of S Sepharose Fast Flow equilibrated in Buffer A. The column was washed with Buffer A then 40% Buffer B (1 M NaCl) was passed through the column until bound protein released at this ionic strength had completely eluted (monitored by UV absorbance at 280 nm). Lactoferrin, appearing as a deep salmon pink band at the top of the column, was eluted with 100% Buffer B. The lactoferrin eluates were dialysed as above and freeze-dried. Lactoferrin concentration in the skim milk was estimated by reversed-phase HPLC (Palmano & Elgar, 2000) after isoelectric precipitation of the caseins and the ratio of added iron to lactoferrin calculated to be 0, 5 and 10 time stoichiometry.

Iron saturation of the isolated freeze-dried lactoferrins (made to ˜12 mg/mL in milliQ water and filtered through 0.2 μm PVDF syringe membranes) was estimated by spectrophotometric titration. Iron saturation was 16%, 47% and 59%, respectively in the lactoferrins isolated from skim milk to which 0 mg, 5 mg and 10 mg ammonium ferrous sulphate had been added.

The breakthrough milk from the batch resin load was acidified to pH 4.6 with HCl to coagulate the caseins. The supernatant whey was recovered by centrifugation and the casein pellet allowed to drain. Iron distribution in the casein and whey samples was measured by ICP-OES. Virtually all the iron not associated with the lactoferrin fraction was associated with the casein fraction.

### EXAMPLE 9

### Iron Loading Using Ammonium Ferrous Sulphate at Different Load Flow Rates and Iron/Lactoferrin Stoichiometries

3 g lactoferrin (Fonterra Co-operative Group) was dissolved into 100 mL fresh skim or commercial trim milk, pH ˜6.7. The milk-lactoferrin solution was passed through 100 mL SPBB resin (2.6×20 cm column) equilibrated in 50 mM NaCl, at 7 mL/min to immobilise the lactoferrin on the resin. Ammonium ferrous sulphate (Fe(NH₄)₂(SO₄)₂.6H₂O) was dissolved as a dry salt in required amounts in 100 mL skim or trim milk and this passed through the resin at flow rates of 1 mL/min, 4 mL/min, or 7 mL/min. A stoichiometry (Fe:Lf) of “1” refers to 2 Fe ions per 1 Lf molecule and requires 10 mg of Fe(NH₄)₂(SO₄)₂.6H₂O per 1 g of Lf. A deepening of the pink colour on the resin (due to the immobilised lactoferrin binding iron) was generally observed with passage of the iron-milk. After loading of the iron-milk, the column was washed with 1 bed volume (bv) (100 mL) 50 mM NaCl followed by elution of bound protein with firstly 0.45M NaCl, then 1.5M NaCl. Lactoferrin was eluted as a discrete fraction, generally intensely red-orange in colour, in the 1.5M NaCl eluate. The resin was then washed with 2 bv 50 mM NaCl followed by cleansing and sanitising with 2 bv 0.1M NaOH.

The degree of iron saturation of lactoferrin in the 1.5M NaCl eluate was assessed by spectrophotometric titration. Iron content of the iron load was confirmed by ICP-OES analysis.

In some cases, the lactoferrin was loaded in 50 mM NaCl as above and the resin ‘conditioned’ with 2-5 bv milk prior to iron loading to simulate conditions where endogenous lactoferrin would be loaded from milk. In yet another variation, the column was equilibrated in milk prior to loading of lactoferrin in milk. The flow rate for all units of operation except iron loading was maintained at 7 mL/min.

Results are given in Table 1 along with the basic conditions utilised for each experiment. Any changes from the basic operating conditions indicated in the column headers are bracketed within the Table.

TABLE 1

| Column | Lf load | Post Lf | Fe load | | | Fe |
|---|---|---|---|---|---|---|
| equili- | Sub- | load | Sub- | Fe load | | satu- |
| bration | strate | Wash | strate | rate | | ration |
| substrate | (1 bv) | (1 bv) | (1 bv) | (mL/min) | Fe:Lf | (%) |
| 50 mM NaCl | milk | 50 mM NaCl | milk | 7 | 1 | 55 |
| Milk (5bv) | milk | none | milk | 7 | 3 | 65 |
| 50 mM NaCl | milk | 50 mM NaCl | milk | 7 | 5 | 76 |
| 50 mM NaCl | 50 mM | milk (5bv) | milk | 4 | 1.5 | 66 |
| | NaCl | | | | | |
| 50 mM NaCl | milk | none | milk | 4 | 2 | 67 |
| Milk (5bv) | milk | none | milk | 4 | 2 | 65 |
| 50 mM NaCl | 50 mM | milk (5bv) | milk | 4 | 2 | 78 |
| | NaCl | | | | | |
| 50 mM NaCl | 50 mM | milk (5bv) | milk | 4 | 2.5 | 75 |
| | NaCl | | | | | |
| 50 mM NaCl | milk | milk (5bv) | milk | 4 | 3 | 67 |
| 50 mM NaCl | 50 mM | milk (5bv) | milk | 4 | 3 | 89 |
| | NaCl | | | | | |
| Milk (5bv) | milk | milk (1bv) | milk | 4 | 4 | 80 |
| Milk (3bv) | milk | none | milk | 4 | 5 | 87 |
| 50 mM NaCl | 50 mM | milk (5bv) | milk | 4 | 6 | 90 |
| | NaCl | | | | | |
| Milk (3bv) | milk | none | milk | 1 | 2 | 85 |
| Milk (3bv) | milk | none | milk | 1 | 3 | 92 |

In Table 1, Fe:Lf refers to the stoichiometry of iron to lactoferrin where a stoichiometry of 1 is just sufficient to completely load a given amount of lactoferrin assuming that lactoferrin is in the apo-state (no bound iron). In other words, a Fe:Lf of 1 refers to a stoichiometry of 1 where 2 metal ions per lactoferrin molecule are provided. The iron saturation of native lactoferrin (no iron load) was estimated at 15%.

The method was successful in saturating lactoferrin to 60% and above when an iron/lactoferrin stoichiometry of 1 or greater was used.

### EXAMPLE 10

### Iron Loading Using Ferrous Sulphate at Different Load Flow Rates and Iron/Lactoferrin Stoichiometries

Experiments were conducted in essentially the same manner as in Example 9, except that ferrous sulphate (FeSO₄.7H₂O) dissolved as a dry salt in milk (or load substrate) was used as the iron donor. For a Fe:Lf stoichiometry of 1 (2 Fe: 1 Lf), 7.1 mg FeSO₄.7H₂O was required per 1 g Lf. Results are shown in Table 2.

TABLE 2

| Column | Lf load | Post Lf | Fe load | | | Fe |
|---|---|---|---|---|---|---|
| equili- | Sub- | load | Sub- | Fe load | | satu- |
| bration | strate | Wash | strate | rate | | ration |
| substrate | (1 bv) | (1 bv) | (1 bv) | (mL/min) | Fe:Lf | (%) |
| Milk (5bv) | milk | none | milk | 7 | 4.5 | 70 |
| | | | (1.5bv) | | | |
| Milk (4bv) | milk | none | milk | 7 | 7.2 | 71 |
| Milk (4bv) | milk | none | milk | 4 | 7.2 | 83 |
| Milk (4bv) | milk | none | milk | 1 | 0.72 | 48 |
| Milk (4bv) | milk | none | milk | 1 | 1.5 | 81 |
| Milk (4bv) | milk | none | milk | 1 | 2 | 83 |
| Milk (4bv) | milk | none | milk | 1 | 3 | 84 |
| Milk (3bv) | milk | none | milk | 1 | 6.7 | 90 |
| Milk (4bv) | milk | none | milk | 1 | 7.2 | 95 |

### EXAMPLE 11

### Iron Loading of Immobilised Lactoferrin Using Multiple Passes of Iron-Substrate

The column described in Example 9 was equilibrated with 4 bv skim milk then 3 g lactoferrin in 100 mL skim milk loaded at a flow rate of 12.5 mL/min. This was followed by 70 mL of the skim milk voided through the column during equilibration, then by several passes of 100 mL skim milk containing 30 mg ammonium ferrous sulphate (added as a dry salt at a Fe:Lf stoichiometry of 1-10 mg of Fe(NH₄)₂(SO₄)₂.6H₂O per 1 g of Lf). For this process, the iron loaded milk exiting the column was recycled through the column but at decreasing flow rate. Cycles were as follows:

(1) Two passes at 12.5 mL/min (no change in colour on column observed)
(2) One pass at 10 mL/min (slight increase in intensity of colour on column)
(3) One pass at 5 mL/min (increase in intensity of colour on column, from salmon pink to red)
(4) One pass at 5 mL/min (further increase in intensity of colour on column)

After loading of the iron-milk, the column was washed with 50 mM NaCl followed by elution of bound protein with firstly 0.45M NaCl, then 1.5M NaCl as for Example 9. Iron saturation of the lactoferrin eluate was 85%.

In another experiment, the column was equilibrated as above, 3 g lactoferrin loaded in 1 bv milk at 10 mL/min and 80 mL skim milk containing 30 mg ammonium ferrous sulphate (stoichiometry of 1) was passed through the column with multiple cycles as above but using 5 passes at a flow rate of 10 mL/min followed by 5 passes at a flow rate of 5 mL/min. After loading of the iron-milk, the column was washed with 50 mM NaCl followed by elution of bound protein with firstly 0.45M NaCl, then 1.5M NaCl as for Example 9. Iron saturation of the lactoferrin eluate was 86%.

In a further experiment, the column was equilibrated as above, 3 g lactoferrin loaded in 1 bv milk at 10 mL/min and 80 mL skim milk containing 60 mg ammonium ferrous sulphate (stoichiometry of 2) passed through the column with multiple cycles as above but using 4 passes at 5 mL/min. After loading of the iron-milk, the column was washed with 50 mM NaCl followed by elution of bound protein with firstly 0.45M NaCl, then 1.5M NaCl as for Example 9. Iron saturation of the lactoferrin eluate was 92%.

### EXAMPLE 12

### Iron Loading of Immobilised Lactoferrin Using Variable Load Volumes of Lactoferrin and Ammonium Ferrous Sulphate

Experiments were conducted in essentially the same manner as in Example 9 except that volume of lactoferrin load substrate and iron load substrate was varied along with Fe:Lf stoichiometries (for a Fe:Lf stoichiometry of 1, 10 mg of Fe (NH₄)₂(SO₄)₂.6H₂O per 1 g of Lf was used) and iron load flow rates. Skim milk was used to equilibrate the column and as the lactoferrin and iron load substrate. Lactoferrin was loaded onto the column at a flow rate of 10 mL/min in all cases. Table 3 summarises data obtained.

TABLE 3

| Column | | | | | | Fe |
|---|---|---|---|---|---|---|
| Equili- | | Post Lf | | Fe load | | satu- |
| bration | Lf load | load | Fe load | rate | | ration |
| Volume | Volume | Wash | Volume | (mL/min) | Fe:Lf | (%) |
| 4bv | 1.25 bv | none | 50 bv | 16 | 1 | 42 |
| 4bv | 1 bv | none | 10 bv | 10 | 2 | 83 |
| 4bv | 1 bv | none | 5 bv | 5 | 2 | 88 |
| 3bv | 2 bv | none | 2 bv | 1 | 2 | 85 |
| 4bv | 3 bv | none | 2 bv | 1 | 2 | 87 |
| 4bv | 3 bv | none | 2 bv | 1 | 2 | 90 |
| 4bv | 2 bv | none | 2 bv | 1 | 2.5 | 100 |
| 4bv | 2 bv | none | 1.5 bv | 5 | 3 | 86 |
| 4bv | 1 bv | none | 2 bv | 5 | 3 | 90 |
| 4bv | 1 bv | none | 3 bv | 5 | 3 | 90 |
| 4bv | 2 bv | none | 2 bv | 1 | 3 | 100 |
| 4bv | 2 bv | none | 2 bv | 5 | 4 | 90 |
| 4bv | 2 bv | none | 3 bv | 5 | 4 | 95 |
| 4bv | 1 bv | none | 1 bv | 2 | 4 | 95 |

### EXAMPLE 13

### Use of Low Bed Volumes (<1) of Iron-Loading Substrate

Experiments were conducted in essentially the same manner as in Example 9. The column was equilibrated in 4 bv skim milk, lactoferrin was loaded in milk and then ammonium ferrous sulphate at varying Fe:Lf stoichiometries dissolved in variable volumes of milk (up to 1 bv) loaded at 0.5-4 mL/min. For a Fe:Lf stoichiometry of 1, 10 mg of Fe(NH₄)₂(SO₄)₂.6H₂O per 1 g of Lf. After loading of the iron-milk, the column was washed with 50 mM NaCl followed by elution of bound protein with 0.45M NaCl, then 1.5M NaCl as for Example 9. Table 4 summarises the results.

TABLE 4

| Column | | | | | | | Fe |
|---|---|---|---|---|---|---|---|
| Equili- | | Post Lf | | Fe load | | | satu- |
| bration | Lf load | load | Fe load | Rate* | | | ration |
| Volume | Volume | Wash | Volume | (mL/min) | | Fe:Lf | (%) |
| 4bv | 2 bv | none | 0.1bv | 0.5 | (20) | 2 | 85 |
| 4bv | 2 bv | none | 0.2bv | 0.5 | (10) | 2 | 84 |
| 4bv | 2bv | none | 0.4bv | 1 | (5) | 2 | 88 |
| 4bv | 2 bv | none | 0.3bv | 1 | (10) | 3 | 85 |
| 4bv | 1 bv | none | 0.4bv | 1 | (7.5) | 3 | 81 |
| 4bv | 1 bv | none | 0.5bv | 1 | (6) | 3 | 91 |
| 4bv | 1bv | none | 0.5bv | 1 | (6) | 3 | 94 |
| 4bv | 1bv | none | 0.5bv | 4 | (6) | 3 | 74 |
| 4bv | 1 bv | none | 0.6bv | 1 | (5) | 3 | 87 |
| 4bv | 1bv | none | 0.72bv | 1 | (4) | 3 | 90 |

*Figures in brackets represent the ratio of iron stoichiometry to load volume of substrate (relative iron concentration)

### EXAMPLE 14

### pH Adjustment of Lactoferrin Eluent

The majority of lactoferrin eluates from the examples above were in the pH range 6.4-6.7. Adjustment of the pH of the eluent (using NaCl) was introduced as an additional step.

Experiments were conducted as in Example 9. The column was equilibrated in 4 bv skim milk, 3 g lactoferrin was loaded in 1 bv skim milk and then ammonium ferrous sulphate at Fe:Lf stoichiometry of 1, 2, 3 or 4 was dissolved in 1 bv of milk and loaded at either 1, 2 or 8 mL/min. Elution was carried out as in Example 9 except that 1% 0.1 M NaOH was added to the NaCl eluent to give a pH of 9-10. Results are presented in Table 5 and compared in one case with a result for a similar experiment in which the NaCl eluent was not pH adjusted.

TABLE 5

| | Fe load rate | Lactoferrin | Fe |
|---|---|---|---|
| Fe:Lf | (mL/min) | eluate pH | saturation (%) |
| 1 | 1 | 7.16 | 61 |
| 2 | 1 | 7.19 | 94 |
| 2 | 1 | 6.62 (na) | 85 |
| 2 | 8 | 6.92 | 80 |
| 3 | 1 | 8.0 | ~100 |
| 4 | 2 | 7.5 | ~100 |

na: not adjusted

### EXAMPLE 15

### Use of Alternative Iron-Loading Substrates

Experiments were conducted as in Example 9 except that the iron salt was dissolved in alternative substrates. The column was equilibrated in 4 bv skim milk and 3 g lactoferrin was loaded onto the column in skim milk. Ammonium ferrous sulphate (10 mg per 1 g Lf for a stoichiometry of 1) was loaded dissolved in milk voided from the column during equilibration (milk BT), a milk/water mix, rennet whey or water. Elution was carried out as in Example 9. Operating parameters along with iron saturation levels obtained from the different loading substrates are given in Table 6.

TABLE 6

| Lf load | Fe load | Fe load | | |
|---|---|---|---|---|
| Substrate | Substrate | Rate | | Fe |
| (& Volume) | (& Volume) | (mL/min) | Fe:Lf | saturation (%) |
| Milk (2bv) | Water (1.5bv) | 5 | 3 | ~65 |
| Milk (1bv) | Milk/water | 1 | 2 | 78 |
| | 50:50 (1bv) | | | |
| Milk (1bv) | Whey (1.5bv) | 5 | 1.5 | 65 |
| Milk (1bv) | Milk BT(1bv) | 2 | 4 | 92 |

The different iron load substrates all enabled iron binding by immobilised lactoferrin. The level of residual iron (measured by ICP-OES) found in the 1.5M NaCl eluate from the water iron loading was significant, indicating iron had exchanged onto the column during loading. Notably, the eluate was a browny orange (rather than red orange) and the UV-Visible spectra irregular.

Table 7 shows that stoichiometric excess of iron loaded in milk substrate is substantially recovered in the breakthrough (BT) milk and wash from iron-loading, whereas with rennet whey and water substrates, this was not the case.

TABLE 7

| | Iron Content (mg) | | | | | |
|---|---|---|---|---|---|---|
| | Milk | Milk | Milk | Milk/H2O | Rennet | Water |
| Substrate | (7x | (4x | (1.5x | (2x | whey | (3x |
| Tested | Fe) | Fe) | Fe) | Fe) | (1.5x Fe) | Fe) |
| Fe Loaded | 26.4 | 16.1 | 6.4 | 8.3 | 6.6 | 11.9 |
| BT + wash | 22.1 | 10.9 | 4.2 | 5.5 | 2 | 1 |
| Lf Eluate | 2.9 | 3.6 | 1.7 | 2.1 | 1.9 | 9.3 |
| Cleaning | ND | 1.4 | ND | NA | 1.6 | 0.3 |
| wash | | | | | | |

Iron measured by ICP-OES. ND = not detected. NA = not analysed.

### EXAMPLE 16

### Loading of an Alternative Metal Ion

The experiment was conducted in essentially the same manner as in Example 9. The column was equilibrated in 4 bv skim milk, then 3 g lactoferrin dissolved in 1 bv skim milk loaded at a flow rate of 8 mL/min. This was followed by 1 bv skim milk containing 142.3 mg cupric sulphate (CuSO4.5H2O) added as a dry salt and at a Cu:Lf stoichiometry of 3:1 (47.4 mg of CuSO₄.5H₂O per 1 g of lactoferrin), with loading at 1 mL/min. Lactoferrin in the 1.5 M NaCl eluate was estimated to be ˜34% saturated with copper by spectrophotometric absorption (Ainscough et al, 1979).

### EXAMPLE 17

### Affinity Resin for Lactoferrin Capture/Immobilisation

The column as in Example 9 was packed with 50 mL Heparin Sepharose 6 Fast Flow™ (Amersham GE), washed with 2M NaCl and equilibrated in 200 mL skim milk. Lactoferrin (1.5 g in 50 mL skim milk) was loaded onto the column at 4 mL/min. 45 mg ammonium ferrous sulphate was dissolved as a dry salt into 50 mL skim milk to give a Fe:Lf stoichiometry of 3 (30 mg of Fe(NH₄)₂(SO4)₂.6H₂O per 1 g of lactoferrin) and this loaded onto the resin at 0.5 mL/min (to give a contact time comparable to the 100 mL column and 1 mL/min Fe-milk loading). The column was washed with 50 mM NaCl followed by elution with 0.45 M NaCl and 1.5 M NaCl (adjusted to pH 7.0 with NaOH). Lactoferrin was recovered in the 1.5 M NaCl eluate and found to be ˜100% saturated with iron.

### INDUSTRIAL APPLICATION

Because of its known health benefits, metal-ion saturated lactoferrin, particularly iron-lactoferrin has applications as a food, drink, nutraceutical or pharmaceutical ingredient.

Those persons skilled in the art will understand that the above description is provided by way of illustration only and that the invention is not limited thereto.

### REFERENCES

Ainscough E W, Brodie A M & Plowman J E (1979). The chromium, manganese, cobalt and copper complexes of human lactoferrin. Inorganica Chimica Acta 33, 149-153.
Bates G W, Billups C, Saltman P (1967). The kinetics and mechanism of iron exchange between chelates and transferrin. 1. The complexes of citrate and nitriloacetic acid. J Biol. Chem 242, 2810-2815.
Bates G W and Schlabach M R (1973). The reaction of Ferric salts with Transferrin. J Biol. Chem 248, 3228-3232.
Blackberg L, Hernell O. Isolation of lactoferrin from human whey by a single chromatographic step. FEBS Lett. 1980 Jan. 14; 109(2):180-3.
Brock J H, Arzabe F R (1976). Cleavage of diferric bovine transferrin into two monomeric fragments. FEBS Lett. 69, 63-66.
Elgar D F, Norris C S, Ayers J S, Pritchard M, Otter D E, Palmano K P. Simultaneous separation and quantitation of the major bovine whey proteins including proteose peptone and caseinomacropeptide by reversed-phase high-performance liquid chromatography on polystyrene-divinylbenzene. J Chromatogr A. 2000 May 12; 878(2):183-96.
Ena J M, Castillo H, Sanchez L, Calvo M. Isolation of human lactoferrin by affinity chromatography using insolubilized bovine beta-lactoglobulin. J Chromatogr. 1990 Feb. 23; 525(2):442-6.
Moore S A, Anderson B F, Groom C R, Haridas M, Baker E N. Three-dimensional structure of diferric bovine lactoferrin at 2.8 A resolution. J Mol Biol. 1997 Nov. 28; 274(2):222-36.
Palmano K P, Elgar D F. Detection and quantitation of lactoferrin in bovine whey samples by reversed-phase high-performance liquid chromatography on polystyrene-divinylbenzene. J Chromatogr A. 2002 Feb. 22; 947(2):307-11.
Scopes, Robert K E (Ed), Protein Purification: Principles and Practice, 3^{rd} Edition, Springer-Verlag, New York, 1994.
Tanaka T, Nakamura I, Lee N Y, Kumura H, Shimazaki K. Expression of bovine lactoferrin and lactoferrin N-lobe by recombinant baculovirus and its antimicrobial activity against Prototheca zopfii. Biochem Cell Biol. 2003 October; 81(5):349-54.

1. A process for preparing metal ion-lactoferrin comprising contacting a lactoferrin source with a lactoferrin-binding matrix to immobilise lactoferrin in the matrix, contacting the immobilised lactoferrin with a source of metal ions or a milk composition comprising a source of metal ions to produce metal ion-lactoferrin and recovering the metal ion-lactoferrin from the matrix.

2. A process of claim 1 comprising contacting the immobilised lactoferrin with a source of metal ions and a milk composition in any order to produce metal ion-lactoferrin and recovering the metal ion-lactoferrin from the matrix.

3. (canceled)

4. (canceled)

5. (canceled)

6. (canceled)

7. A process for preparing metal ion-lactoferrin comprising:
(a) contacting a column having a column volume and comprising a lactoferrin-binding matrix with a lactoferrin source to immobilise the lactoferrin in the matrix,
(b) contacting the immobilised lactoferrin with a source of metal ions or with at least one column volume or part thereof of a milk composition comprising a source of metal ions to produce metal ion-lactoferrin, and
(c) recovering the metal ion-lactoferrin from the matrix.

8. A process of claim 7 comprising
(a) contacting the immobilised lactoferrin with a source of metal ions and a milk composition in any order to produce metal ion-lactoferrin, and
(b) recovering the metal ion-lactoferrin from the matrix.

9. (canceled)

10. (canceled)

11. (canceled)

12. A process for preparing metal ion-lactoferrin comprising
(a) contacting a membrane, optionally comprising a lactoferrin-binding matrix with a lactoferrin source to immobilise the lactoferrin on the membrane or in the matrix,
(b) contacting the immobilised lactoferrin with a source of metal ions to produce metal ion-lactoferrin, and
(c) recovering the metal ion-lactoferrin from the membrane or matrix.

13. A process of claim 12 comprising:
(a) contacting a membrane, optionally comprising a lactoferrin-binding matrix with milk or a derivative thereof containing lactoferrin to immobilise the lactoferrin on the membrane or in the matrix,
(b) contacting the immobilised lactoferrin with an amount of the milk or derivative thereof that further comprises a source of metal ions to produce metal ion-lactoferrin, and
(c) recovering the metal ion-lactoferrin from the membrane or matrix.

14. A process of claim 1 wherein the lactoferrin source is aqueous.

15. A process of claim 1 wherein the lactoferrin source is milk or a derivative thereof.

16. A process of claim 1 wherein the lactoferrin source is selected from a substantially pure lactoferrin composition, a crude lactoferrin composition, recombined or fresh whole milk, recombined or fresh skim milk, reconstituted whole or skim milk powder, skim milk concentrate, skim milk retentate, concentrated milk, buttermilk, ultrafiltered milk retentate, milk protein concentrate (MPC), milk protein isolate (MPI), calcium depleted milk protein concentrate (MPC), low fat milk, low fat milk protein concentrate (MPC), colostrum, a colostrum fraction, colostrum protein concentrate (CPC), colostrum whey, whey, whey protein isolate (WPI), whey protein concentrate (WPC), sweet whey, lactic acid whey, mineral acid whey, salt whey, reconstituted whey powder, any milk or colostrum processing stream comprising whey proteins, the retentate or permeate comprising whey proteins obtained by ultrafiltration or microfiltration of any milk or colostrum processing stream, or the breakthrough or adsorbed fraction comprising whey proteins obtained by chromatographic separation of any milk or colostrum processing stream.

17. A process of claim 15 wherein the milk or derivative thereof containing lactoferrin is selected from the group comprising recombined or fresh whole milk, recombined or fresh skim milk, reconstituted whole or skim milk powder, skim milk concentrate, skim milk retentate, concentrated milk, buttermilk, ultrafiltered milk retentate, milk protein concentrate (MPC), milk protein isolate (MPI), calcium depleted milk protein concentrate (MPC), low fat milk, low fat milk protein concentrate (MPC), colostrum, a colostrum fraction, colostrum protein concentrate (CPC), colostrum whey, whey, whey protein isolate (WPI), whey protein concentrate (WPC), sweet whey, lactic acid whey, mineral acid whey, salt whey, reconstituted whey powder, any milk or colostrum processing stream comprising whey proteins, the retentate or permeate comprising whey proteins obtained by ultrafiltration or microfiltration of any milk or colostrum processing stream, or the breakthrough or adsorbed fraction comprising whey proteins obtained by chromatographic separation of any milk or colostrum processing stream.

18. A process of claim 1 wherein the milk composition is selected from the group comprising recombined or fresh whole milk, recombined or fresh skim milk, reconstituted whole or skim milk powder, skim milk concentrate, skim milk retentate, concentrated milk, buttermilk, ultrafiltered milk retentate, milk protein concentrate (MPC), milk protein isolate (MPI), calcium depleted milk protein concentrate (MPC), low fat milk, low fat milk protein concentrate (MPC), colostrum, a colostrum fraction, colostrum protein concentrate (CPC), colostrum whey, whey, whey protein isolate (WPI), whey protein concentrate (WPC), sweet whey, lactic acid whey, mineral acid whey, salt whey, reconstituted whey powder, from any milk or colostrum processing stream, the permeate obtained by ultrafiltration or microfiltration of any milk or colostrum processing stream, or the breakthrough or adsorbed fraction obtained by chromatographic separation of any milk or colostrum processing stream.

19. A process of claim 1 wherein the source of metal ions is a metal ion salt.

20. A process of claim 19 wherein the salt is an ammonium citrate salt, ammonium sulphate salt, citrate salt, chloride salt, lactate salt, nitrate salt, sulphate salt or a mixture thereof.

21. A process of claim 1 to wherein the metal ions are bismuth ions, chromium ions, cobalt ions, cuprous ions, cupric ions, ferric ions, ferrous ions, manganese ions, zinc ions or mixtures thereof.

22. A process of claim 1 wherein the metal ion concentration of the source of metal ions or the milk composition is sufficient to allow stoichiometric binding of the metal ion by the immobilised lactoferrin.

23. A process of claim 22 wherein the molar ratio of immobilised lactoferrin to metals ions is from about 1:1 to about 1:22.

24. A process of claim 1 wherein the lactoferrin-binding matrix is selected from an ion exchange matrix, an anion exchange matrix, a weak anion exchange matrix, a cation exchange matrix, a weak cation exchange matrix, a strong cation exchange matrix, an affinity matrix, a heparin affinity matrix, a beta-lactoglobulin affinity matrix, an antibody affinity matrix, a metal ion-affinity matrix, a lactoferrin-ligand affinity matrix, an exclusion matrix, a mixed-mode matrix, a microfiltration membrane and an ultrafiltration membrane.

25. (canceled)

26. A process of claim 7 wherein the lactoferrin source or the milk or milk derivative containing lactoferrin is contacted with matrix at a flow rate of about 0.5 to about 20 cv/hr.

27. A process of claim 7 wherein the source of metal ions or milk composition comprising a source of metal ions is contacted with immobilised lactoferrin at a flow rate of about 0.25 to about 10 cv/hr.

28. A process of claim 7 wherein the volume of the source of metal ions or milk composition comprising a source of metal ions is about 0.25, 0.5, 0.75, 1, 1.25, 1.5, 1.75, 2, 2.25, 2.5, 2.75, 3, 3.25, 3.5, 3.75 or 4 column volumes.

29. A process of claim 7 wherein the source of metal ions or the milk composition comprising a source of metal ions is recycled and contacted with the immobilised lactoferrin at least another 1 to 10 times.

30. A process of claim 7 wherein the metal ion-lactoferrin is recovered by contacting the lactoferrin-binding matrix with a solution having a salt concentration of at least about 0.4 M.

31. (canceled)

32. (canceled)

33. A process of claim 7 wherein the pH of metal ion-lactoferrin recovered from the matrix is adjusted to about pH 7.0, 7.5, 8.0, 8.5, 9.0, 9.5 or 10.

34. (canceled)

35. (canceled)

36. A food, drink, food additive, drink additive, dietary supplement, nutritional product, medicament, pharmaceutical or neutraceutical comprising metal ion-lactoferrin produced according to a process as claimed in claim 7.
